(19) United States
(12) Patent Application Publication
(10) Pub. No.: US 2021/0304880 A1
(43) Pub. Date: Sep. 30, 2021

(54) SYSTEM AND METHOD FOR DIRECT PHYSICIAN COMMUNICATION

(71) Applicant: Optiks Solutions, Inc., Piscataway, NJ (US)

(72) Inventors: Anupam NANDWANA, Piscataway, NJ (US); Edward VAZ, Piscataway, NJ (US)

(21) Appl. No.: 17/214,800

(22) Filed: Mar. 27, 2021

Related U.S. Application Data
(60) Provisional application No. 63/000,493, filed on Mar. 27, 2020.

(51) Int. Cl. G16H 40/20; G16H 40/67; G16H 70/40; G16H 50/20; G06F 9/451

(52) U.S. Cl. CPC G16H 40/20; G16H 40/67; G16H 80/00; G16H 50/20; G06F 9/451; G16H 70/40

(57) ABSTRACT

A system and method for securing communication for medical personnel that provides a single channel, to enable the personnel to rapidly reach resources, information or services.

### FIELD OF THE INVENTION

The present invention is of a system and method for direct communication, and in particular, of such a system and method for such a direct communication process that supports easy access to services and resources, yet is also secure.

### BACKGROUND OF THE INVENTION

Physicians and other medical personnel often need rapid access to information and other resources, as well as to various services. For example, they may need information on a new medical device, biological or pharmaceutical. They may need to report an adverse event. Other types of information and services are also important to be able to access. However, currently medical personnel do not have a single, easy and secure way to access such resources, information or services.

### BRIEF SUMMARY OF THE INVENTION

The present invention overcomes the drawbacks of the background art by providing a system and method for securing communication for medical personnel that provides a single channel, to enable the personnel to rapidly reach resources, information or services.

Implementation of the method and system of the present invention involves performing or completing certain selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

Unless otherwise defined, all technical and scientific terms used herein have the same meaning as commonly understood by one of ordinary skill in the art to which this invention belongs. The materials, methods, and examples provided herein are illustrative only and not intended to be limiting.

An algorithm as described herein may refer to any series of functions, steps, one or more methods or one or more processes, for example for performing data analysis.

Implementation of the apparatuses, devices, methods and systems of the present disclosure involve performing or completing certain selected tasks or steps manually, automatically, or a combination thereof. Specifically, several selected steps can be implemented by hardware or by software on an operating system, of a firmware, and/or a combination thereof. For example, as hardware, selected steps of at least some embodiments of the disclosure can be implemented as a chip or circuit (e.g., ASIC). As software, selected steps of at least some embodiments of the disclosure can be implemented as a number of software instructions being executed by a computer (e.g., a processor of the computer) using an operating system. In any case, selected steps of methods of at least some embodiments of the disclosure can be described as being performed by a processor, such as a computing platform for executing a plurality of instructions.

Software (e.g., an application, computer instructions) which is configured to perform (or cause to be performed) certain functionality may also be referred to as a “module” for performing that functionality, and also may be referred to a “processor” for performing such functionality. Thus, processor, according to some embodiments, may be a hardware component, or, according to some embodiments, a software component.

Further to this end, in some embodiments: a processor may also be referred to as a module; in some embodiments, a processor may comprise one or more modules; in some embodiments, a module may comprise computer instructions—which can be a set of instructions, an application, software—which are operable on a computational device (e.g., a processor) to cause the computational device to conduct and/or achieve one or more specific functionality. Some embodiments are described with regard to a “computer,” a “computer network,” and/or a “computer operational on a computer network.” It is noted that any device featuring a processor (which may be referred to as “data processor”; “pre-processor” may also be referred to as “processor”) and the ability to execute one or more instructions may be described as a computer, a computational device, and a processor (e.g., see above), including but not limited to a personal computer (PC), a server, a cellular telephone, an IP telephone, a smart phone, a PDA (personal digital assistant), a thin client, a mobile communication device, a smart watch, head mounted display or other wearable that is able to communicate externally, a virtual or cloud based processor, a pager, and/or a similar device. Two or more of such devices in communication with each other may be a “computer network.”

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the drawings:

FIGS. 1A-1B show non-limiting, exemplary illustrations of various systems according to embodiments of the present invention;

FIGS. 2A-2C relate to non-limiting, exemplary illustrations of various dashboard implementations according to embodiments of the present invention;

FIG. 3 shows a non-limiting communication flow which may be performed after one of the above action icons is selected;

FIG. 4 shows a non-limiting, exemplary workflow for advertising displays on the previously described dashboard, to the medical user;

FIG. 5 shows a non-limiting, exemplary workflow for providing alerts through the previously described dashboard, to the medical user; and

FIG. 6 shows a non-limiting, exemplary drug information dashboard.

### DESCRIPTION OF AT LEAST SOME EMBODIMENTS

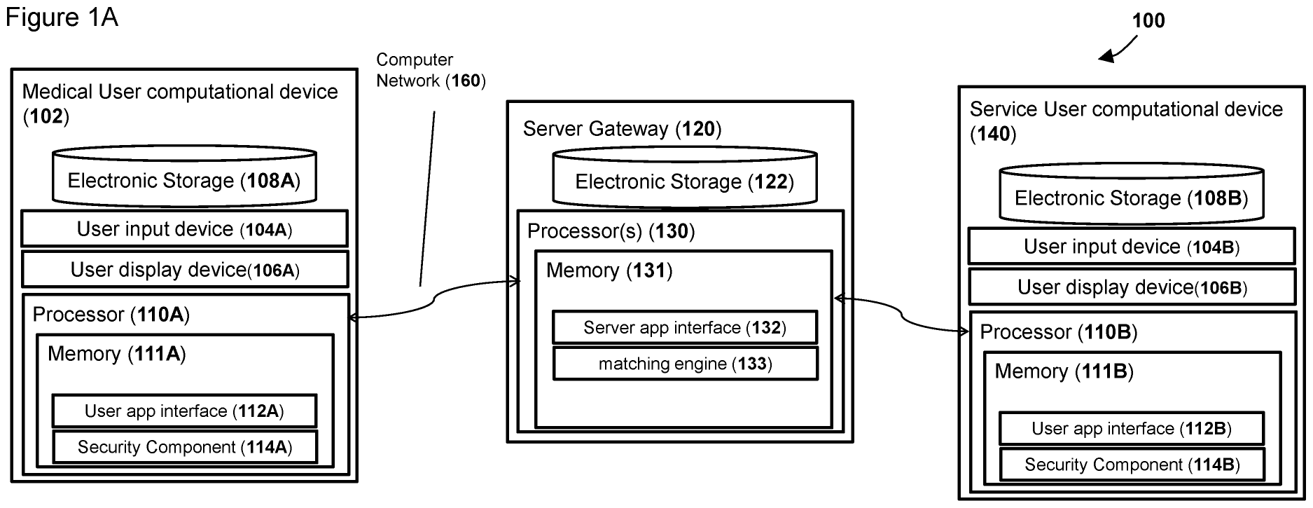

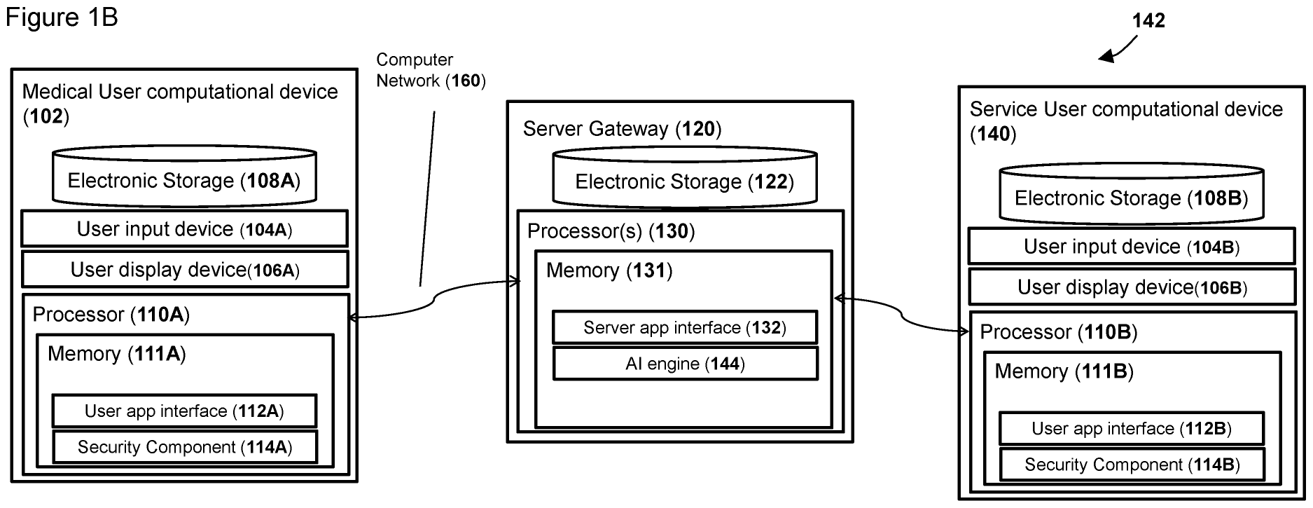

FIG. 1A illustrates a system 100 configured for supporting communication between a medical user, such as a physician or other medical personnel, and a service provider, such as a pharmaceutical, or other medical product or service company, in accordance with one or more implementations of the present invention.

In some implementations, the system 100 may include a medical user computational device 102 and a server gateway 120 that communicates with the medical user computational device 102 through a computer network 160, such as the internet. (“Server gateway” and “server” are equivalent and may be used interchangeably). A user may access the system 100 via medical user computational device 102.

The medical user computational device 102 features a user input device 104A, a user display device 106A, an electronic storage 108A (or user memory), and a processor 110A (or user processor). The medical user computational device 102 may optionally comprise one or more of a desktop computer, laptop, PC, mobile device, cellular telephone, and the like.

The user input device 104A allows a user to interact with the medical user computational device 102. Non-limiting examples of a user input device 104A are a keyboard, mouse, other pointing device, touchscreen, and the like.

The user display device 106A displays information to the user. Non-limiting examples of a user display device 106A are computer monitor, touchscreen, and the like. The user input device 104A and user display device 106A may optionally be combined to a touchscreen, for example.

The electronic storage 108A may comprise non-transitory storage media that electronically stores information. The electronic storage media of electronic storage 108A may include one or both of system storage that is provided integrally (i.e., substantially non-removable) with a respective component of system 100 and/or removable storage that is removably connected to a respective component of system 100 via, for example, a port (e.g., a USB port, a firewire port, etc.) or a drive (e.g., a disk drive, etc.). The electronic storage 108A may include one or more of optically readable storage media (e.g., optical discs, etc.), magnetically readable storage medium (e.g., flash drive, etc.), and/or other electronically readable storage medium. The electronic storage 108A may include one or more virtual storage resources (e.g., cloud storage, a virtual private network, and/or other virtual storage resources). The electronic storage 108A may store software algorithms, information as determined by processor 110A, and/or other information that enables components of a system 100 to function as described herein.

The processor 110A refers to a device or combination of devices having circuitry used for implementing the communication and/or logic functions of a particular system. For example, a processor may include a digital signal processor device, a microprocessor device, and various analog-to-digital converters, digital-to-analog converters, and other support circuits and/or combinations of the foregoing. Control and signal processing functions of the system are allocated between these processing devices according to their respective capabilities. The processor may further include functionality to operate one or more software programs based on computer-executable program code thereof, which may be stored in a memory. As the phrase is used herein, the processor may be “configured to” perform a certain function in a variety of ways, including, for example, by having one or more general-purpose circuits perform the function by executing particular computer-executable program code embodied in computer-readable medium, and/or by having one or more application-specific circuits perform the function.

The processor 110A is configured to execute readable instructions 111A stored in a memory. The computer readable instructions 111A include instructions for providing a user app interface 104A, security component 114A, and/or other components when executed by processor 110A.

The user app interface 104A provides a user interface presented via the medical user computational device 102. The user app interface 104A may be a graphical user interface (GUI). The user interface may provide information to the user. In some implementations, the user interface may present information associated with one or more transactions. The user interface may receive information from the user. In some implementations, the user interface may receive user instructions to perform a transaction. The user instructions may include a selection of a transaction, a command to perform a transaction, and/or information associated with a transaction.

Referring now to server gateway 120 depicted in FIG. 1A, the server gateway 120 communicates with the medical user computational device 102 and a service user computational device 140. The server gateway 120 features an electronic storage 122 (or server memory), one or more processor(s) 130 (or server processor), and/or other components. The server gateway 120 may include a plurality of hardware, software, and/or firmware components operating together to provide the functionality attributed herein to server gateway 120.

The electronic storage 122 may comprise non-transitory storage media that electronically stores information. The electronic storage media of electronic storage 122 may include one or both of system storage that is provided integrally (i.e., substantially non-removable) with a respective component of system 100 and/or removable storage that is removably connected to a respective component of system 100 via, for example, a port (e.g., a USB port, a firewire port, etc.) or a drive (e.g., a disk drive, etc.). The electronic storage 122 may include one or more of optically readable storage media (e.g., optical discs, etc.), magnetically readable storage medium (e.g., flash drive, etc.), and/or other electronically readable storage medium. The electronic storage 122 may include one or more virtual storage resources (e.g., cloud storage, a virtual private network, and/or other virtual storage resources). The electronic storage 122 may store software algorithms, information as determined by processor 130, and/or other information that enables components of a system 100 to function as described herein.

The processor 130 may be configured to provide information processing capabilities in server gateway 120. As such, the processor 130 may include a device or combination of devices having circuitry used for implementing the communication and/or logic functions of a particular system. For example, a processor may include a digital signal processor device, a microprocessor device, and various analog-to-digital converters, digital-to-analog converters, and other support circuits and/or combinations of the foregoing. Control and signal processing functions of the system are allocated between these processing devices according to their respective capabilities. The processor may further include functionality to operate one or more software programs based on computer-executable program code thereof, which may be stored in a memory. As the phrase is used herein, the processor may be “configured to” perform a certain function in a variety of ways, including, for example, by having one or more general-purpose circuits perform the function by executing particular computer-executable program code embodied in computer-readable medium, and/or by having one or more application-specific circuits perform the function.

The processor 130 is configured to execute machine-readable instructions stored in a memory 131. The machine-readable instructions include instructions for a server app interface 132, and/or other components when executed by processor 130.

Service user computational device 140 features similar components to those shown at medical user computational device 102, but indicated with “B” as a reference rather than “A”. Service user computational device 140 enables one or more services to be provided to the medical user, which can be requested through medical user computational device 102. For example, the medical user may request information about a new product, such as a new pharmaceutical. This request is submitted through user app interface 112A and is then passed to server app interface 132. The request is passed to user app interface 112B. The service user may then start a process to provide the requested information electronically to medical user computational device 102, in which case it may be passed to user app interface 112A. The information may be transmitted synchronously, in the form of a “chat”, or asynchronously.

Alternatively, a direct video link may be made between medical user computational device 102 and service user computational device 140, whether through user app interfaces 112A and 112B, or through another type of software that supports such a direct video link. As another alternative, the medical user may request that physical product literature or a physical product be sent to a physical address associated with the medical user, through user app interface 112A.

Preferably, as described in greater detail below, user app interface 112A shows a dashboard of a plurality of service providers, such as a plurality of companies for example. The medical user may select a service provider by clicking a button or otherwise indicating the service provider through user app interface 112A. Next, preferably the medical user is able to select a specific type of action, again through user app interface 112A. Optionally, as described in greater detail below, alerts or other information may be displayed through user app interface 112A.

For greater security, optionally a secure channel is created between user app interface 112A and user app interface 112B, through security components 114A and 114B, respectively. For example, security components 114A and 114B may support encrypted communication. Such encryption may also allow communication to pass securely through server app interface 132, while maintaining both data security and also privacy of the data.

Server gateway 120 may also comprise a matching engine 133 for routing communication correctly between medical user computational device 102 and service user computational device 140. For example, matching engine 133 may connect a request from medical user computational device 102 to the correct service user computational device 140.

Preferably, each of memory 111A, 111B and 131 is configured for storing a defined native instruction set of codes. Each of processor 110A, 110B and 130 is configured to perform a defined set of basic operations in response to receiving a corresponding basic instruction selected from the defined native instruction set of codes stored in each of memory 111A, 111B and 131, respectively.

For example and without limitation, memory 111A may store a first set of machine codes selected from the native instruction set for receiving user requests through user app interface 112A; a second set of machine codes selected from the native instruction set for communicating such requests to and receiving responses from server gateway 120; and a third set of machine codes selected from the native instruction set for supporting encrypted communication with service user computational device 140 through security components 114A and 114B. Memory 111B may have a similar, equivalent set of machine codes.

Memory 111A also preferably stores a fourth set of machine codes selected from the native instruction set for providing a dashboard with “one click” communication capabilities with medical suppliers. Memory 111A also preferably stores a fifth set of machine codes selected from the native instruction set for providing a dashboard with rapid drug information retrieval capabilities.

For example and without limitation, memory 131 may store a first set of machine codes selected from the native instruction set for receiving user requests from medical user computational device 102; a second set of machine codes selected from the native instruction set for determining the correct communication response, drug information and/or connection; and a third set of machine codes selected from the native instruction set for providing the correct communication response, drug information and/or connection to medical user computational device 102. A similar set of codes may be provided to support communication with service user computational device 140.

FIG. 1B shows a similar system as for the system of FIG. 1A, but with the addition of an AI engine for performing more detailed matching between medical user needs and service providers. The AI engine 144 may include machine learning and/or deep learning algorithms, which is explained later in greater detail. AI engine 144 may for example locate an appropriate service provider computational device related to an appropriate service provider, if the medical user does not already have a specific identified service provider to which the request should be sent. Doctors can ask for specific companies or for specific types of companies, after which AI engine 144 provides a match. AI engine 144 is a non-limiting example of a matching service or matching engine. Each different resource, type of information or service may be provided as a menu item. Alternatively or additionally, the initial connection may be based on company name, brand name, or function. Optionally AI engine 144 begins by matching the company, then shows available services for that company.

The operations of AI engine 144 may be supported by a fourth set of machine codes selected from the native instruction set and stored in memory 131.

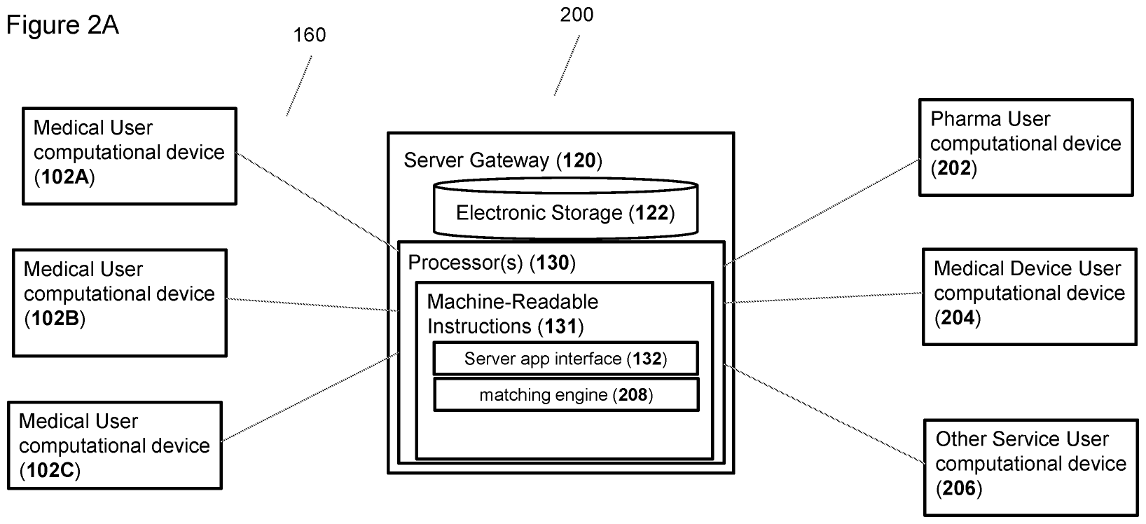

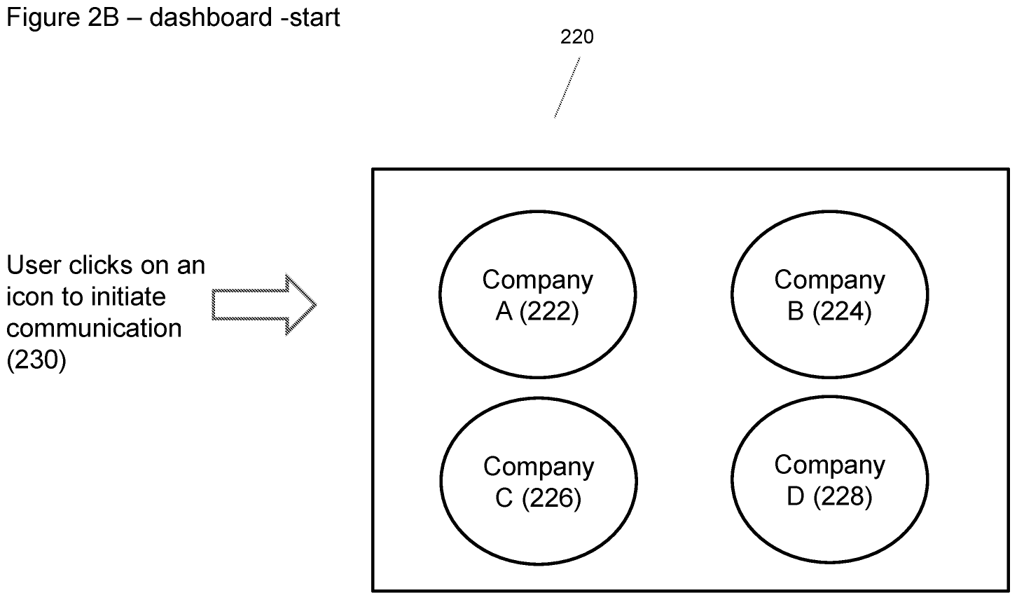

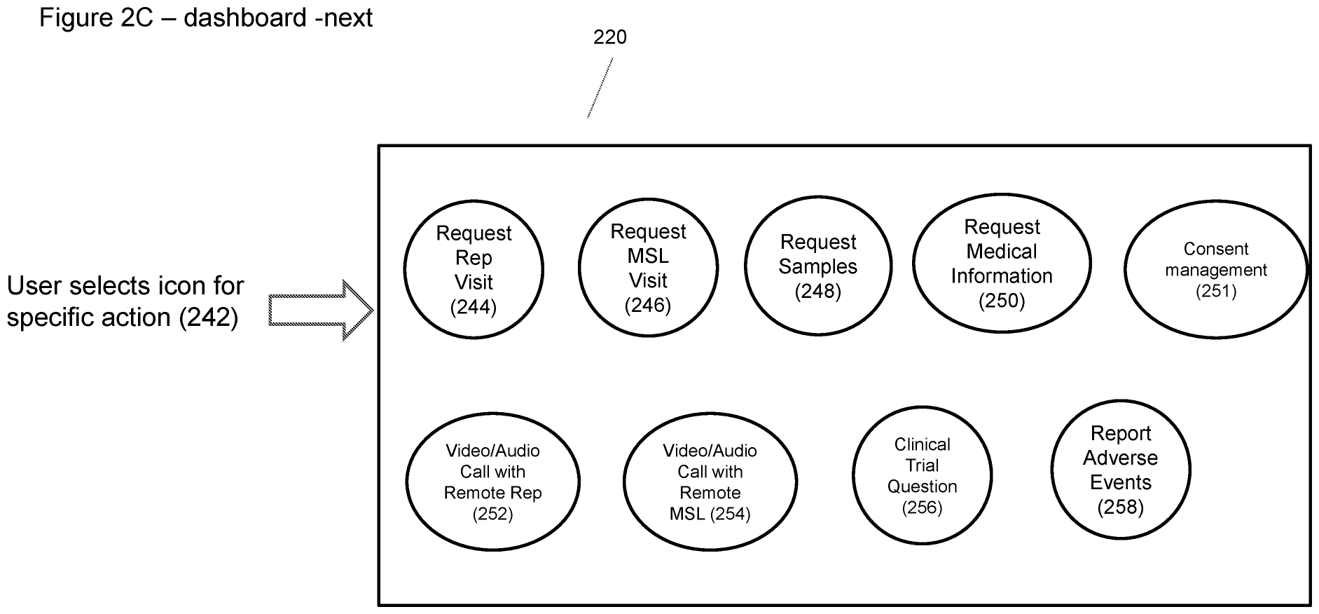

FIG. 2A shows a system with a plurality of different medical users, connected to different medical user computational devices, who wish to obtain services from a plurality of different service providers. Various components that were shown in FIGS. 1A and 1B are not shown for the sake of clarity. As shown in a system 200, a plurality of medical user computational devices 102A, 102B and 102C communicate with a plurality of different service providers through server gateway 120. The different service providers are shown as communicating through pharma user computational device 202, a medical device user computational device 204, and a general other service provider user computational device 206.

FIG. 2B shows a schematic diagram of a dashboard 220, featuring a plurality of icons for a plurality of different companies, of which four are shown for the sake of illustration and without any intention of being limiting. Dashboard 220 is displayed on a medical user computational device (not shown). Icons are shown as icon 222 for Company A, icon 224 for Company B, icon 226 for Company C and icon 228 for Company D. In a process 230, the medical user requests communication with one of the companies by selecting the requisite icon 222-228 on dashboard 220, for example by clicking on it.

FIG. 2C shows an optional next display on the dashboard, which may provide a plurality of additional actions for the medical user to select. Dashboard 220 now enables the medical user to select an icon for a specific action at 242. The actions preferably relate to various service options offered by the company whose icon was selected previously. An MSL as used below is a medical science liaison (a type of company representative or agent who is knowledgeable about the drug and about the science).

The actions may include but are not limited to one or more of Request Representative (Rep) Visit (244), Request MSL Visit (246), Request Samples (248), Request Medical Information (250), Consent management (251), Video/Audio Call with Remote Rep (252), Video/Audio Call with Remote MSL (254), Clinical Trial Question (256) and/or Report Adverse Events (258).

Consent management 251 relates to a plurality of communication permissions that the medical user grants. Such permissions may be simple or complex. For example, the permissions may only relate to certain drugs about which the medical user wishes to receive information. Alternatively or additionally, the medical user may permit any type of email communication but not in person visits, or may permit sending a representative to the office under specific conditions. Various types of blanket permissions may also be given. Overall these permissions relate to the relationship that the medical user desires to have with the company.

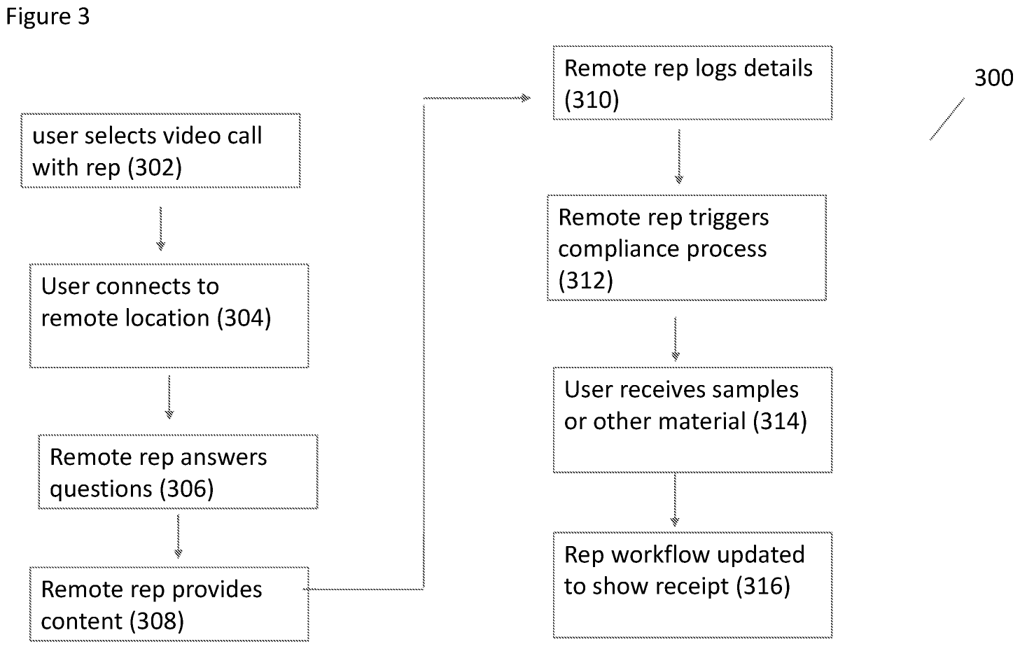

FIG. 3 shows a non-limiting communication flow which may be performed after one of the above action icons is selected. In this non-limiting example, the medical user wishes to initiate a video call with a remote representative. More generally, upon clicking on one of the service options, medical professionals will be led to a detailed service option. These icons will be connected to a dedicated application instance in life sciences companies on their own or contracted environment. Usage flows will be defined using custom workflow depending upon the business and product.

As shown in FIG. 3, in a flow 300, the medical user first selects the action of a video call with a remote representative in 302. At 304, the medical user connects to the remote location, for example by using readily available calling services such as Microsoft Teams, Zoom Meeting and the like. At 306, the remote representative communicates with the medical user, for example by answering questions. The remote representative for example discusses the advantages of the medication or discusses any questions the physician or other medical user may have.

At 308, the remote representative may for example provide content to the medical user. The content, which may be medical, marketing or other content, may be shared in any suitable format, including but not limited to video, audio, text and/or print literature formats.

At 310, the remote representative may log details of the discussion in a cloud hosted application with the medical user information. Such logging both supports follow up and may be required for compliance. The remote representative preferably executes a workflow to trigger required compliance processes at 312. At 314, the medical user receives samples or other material, for example by courier, and preferably signs for them. At 316, the remote representative workflow is updated to show receipt.

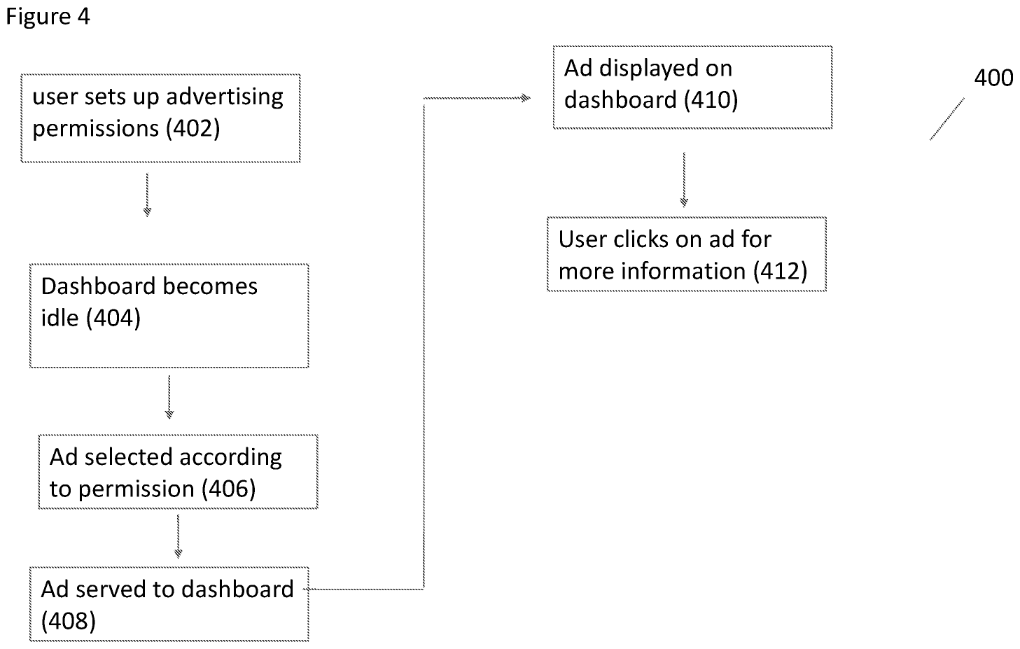

FIG. 4 shows a non-limiting, exemplary workflow for advertising displays on the previously described dashboard, to the medical user. As shown in a flow 400, the medical user sets up advertising permissions at 402, for example by indicating the types of products and/or services the user wishes to view advertising for. At 404, the dashboard becomes idle, for example because the medical user is not using it at the moment. At 406, an advertisement is selected according to the previously determined user permissions, which may be performed according to any known advertising technology (adtech). At 408, the advertisement is served to the dashboard and at 410, it is displayed on the dashboard. Optionally, at 412, the user clicks on the displayed advertisement for more information.

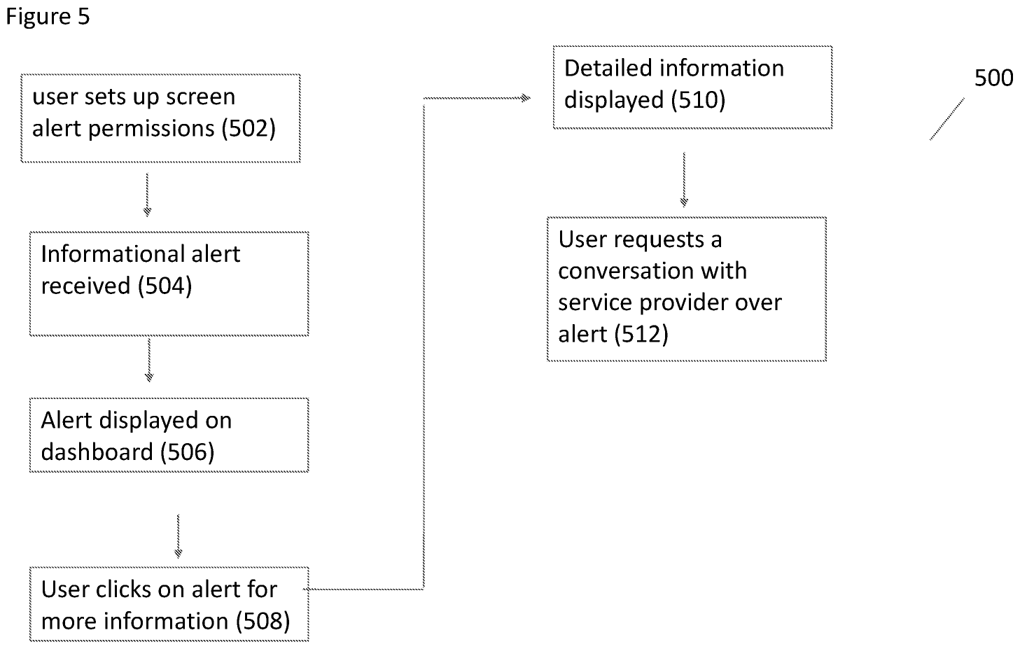

FIG. 5 shows a non-limiting, exemplary workflow for providing alerts through the previously described dashboard, to the medical user. As shown in a flow 500, the medical user sets up screen alert permissions at 502, for example by indicating the types of alerts that the medical user wishes to receive. At 504, an information alert is received. At 506, the alert is displayed on the dashboard. At 508, the user clicks on the displayed alert for more information. At 510, the detailed alert information is displayed. At 512, the user requests a conversation with a service provider over alert.

For any of the embodiments as described herein, optionally the dashboard is provided through a personal computer or mobile device, or on dedicated hardware. Optionally the dashboard connects to a known CRM (customer relationship management) platform, including for example and without limitation Salesforce.

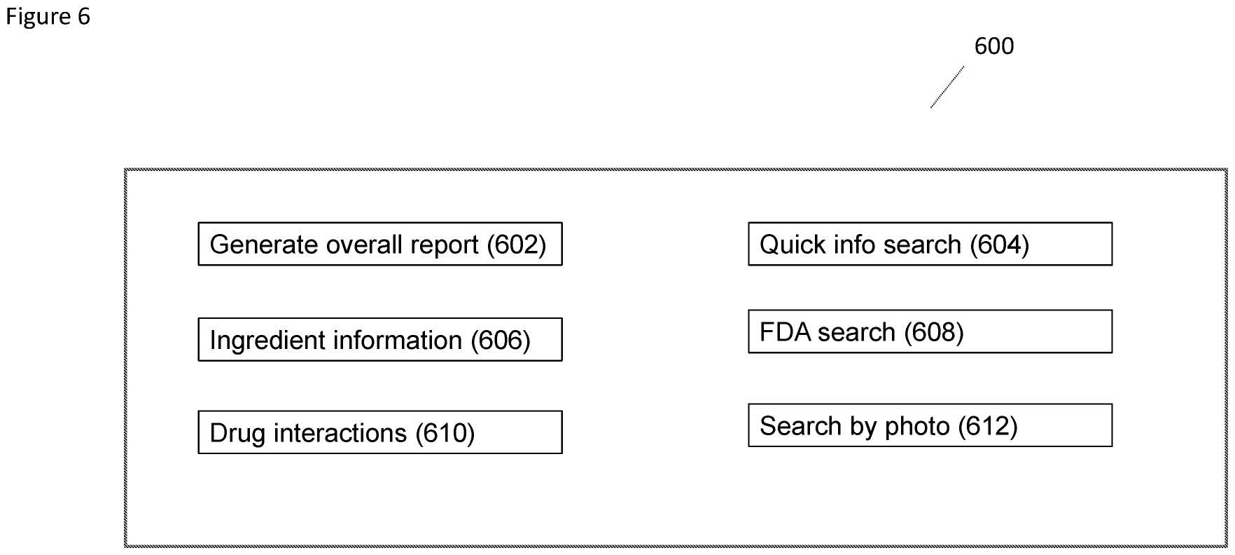

FIG. 6 shows a non-limiting, exemplary drug information dashboard. As shown, a drug information dashboard 600 supports querying the system to find information for any drug using text, image or voice-based search. The information may be stored in the FDA database or any other suitable drug information database, including for example data provided from the drug manufacturer. In this non-limiting example, dashboard 600 includes an option to generate an overall report, for example with a detailed combination of information from other sources at 602; a quick information search option at 604; an ingredient information search option at 606; an FDA database search option at 608; a drug interactions search at 610; and a photo/image based search option at 612.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A system for supporting a direct connection between medical personnel and a remote resource, comprising a computer network, a medical user computational device, wherein the device is controllable by a medical user, the medical user computational device comprising a computer network connection to the computer network and a medical user interface, the medical user interface receiving a request for a direct connection to the remote resource; a server for receiving the request for the direct connection through the computer network; and a resource computational device for receiving a notification of the request for the direct connection from the server, wherein the resource computational device comprises at least one medical resource.

2. The system of claim 1, wherein the at least one resource comprises a service, wherein the resource computational device comprises a service provider computational device, and wherein the service provider is selected from the group consisting of pharmaceutical, medical device, other medical product and other medical service company.

3. The system of claim 2, wherein the resource computational device comprises a plurality of resource computational devices and wherein the server comprises a matching engine for selecting the resource computational device.

4. The system of claim 3, wherein the resource computational device comprises a plurality of resource computational devices and wherein the medical user interface comprises a dashboard, said dashboard comprising a plurality of direct connectors, each direct connector directly and securely connecting the medical user computational device to one of said plurality of resource computational devices.

5. The system of claim 3, wherein said medical user computational device comprises a memory configured for storing a defined native instruction set of codes and a processor configured to perform a defined set of basic operations in response to receiving a corresponding basic instruction selected from the defined native instruction set of codes stored in said memory, wherein said memory stores a first set of machine codes selected from the native instruction set for receiving user requests through said medical user interface; a second set of machine codes selected from the native instruction set for communicating such requests to and receiving responses from said server gateway; and a third set of machine codes selected from the native instruction set for supporting encrypted communication with said service user computational device through security components executed on each of said medical user computational device and said service user computational device.

6. The system of claim 5, wherein said memory stores a fourth set of machine codes selected from the native instruction set for providing a dashboard with “one click” communication capabilities with said service user computational device through said server gateway.

7. The system of claim 6, wherein said memory stores a fifth set of machine codes selected from the native instruction set for providing a dashboard with rapid drug information retrieval capabilities according to communication with said server gateway.

8. The system of claim 7, wherein said server gateway comprises a memory configured for storing a defined native instruction set of codes and a processor configured to perform a defined set of basic operations in response to receiving a corresponding basic instruction selected from the defined native instruction set of codes stored in said memory, wherein said memory stores a first set of machine codes selected from the native instruction set for receiving user requests from said medical user computational device; a second set of machine codes selected from the native instruction set for determining the correct communication response, drug information and/or connection; and a third set of machine codes selected from the native instruction set for providing the correct communication response, drug information and/or connection to said medical user computational device.

9. The system of claim 8, wherein said matching engine comprises an AI engine, wherein operations of said AI engine are supported by a fourth set of machine codes selected from the native instruction set and stored in said memory of said server.

10. The system of claim 2, wherein said service provider computational device provides content to the medical user computational device through said server gateway, wherein said content is shared in a format selected from the group consisting of video, audio, text and/or print literature formats.

11. The system of claim 2, wherein said service provider computational device and said medical user computational device engage in communication in a format selected from the group consisting of audio call, asynchronous audio, video call, asynchronous video, email and chat.

12. The system of claim 11, wherein said service provider computational device logs details of said communication at said server gateway.

13. The system of claim 12, wherein upon a request from said medical user computational device, said service provider computational device executes a workflow to trigger a required compliance process, wherein said workflow comprises sending physical samples to a physical address associated with said medical user computational device.
